(19) United States
(12) Patent Application Publication
(10) Pub. No.: US 2015/0224278 A1
(43) Pub. Date: Aug. 13, 2015

(54) NEBULIZED VENTILATION SYSTEM

(71) Applicant: PNEUMOFLEX SYSTEMS, LLC, Melbourne, FL (US)

(72) Inventors: W. Robert Addington, Melbourne Beach, FL (US); Stuart P. Miller, Indialantic, FL (US)

(73) Assignee: PNEUMOFLEX SYSTEMS, LLC, Melbourne, FL (US)

(21) Appl. No.: 14/176,370

(22) Filed: Feb. 10, 2014

(51) Int. Cl. A61M 16/14; A61M 15/00; A61M 16/20; A61M 16/16; A61M 16/00; A61M 16/08

(52) U.S. Cl. CPC A61M 16/147; A61M 16/0012; A61M 16/0816; A61M 16/0875; A61M 16/20; A61M 16/16; A61M 15/0086

(57) ABSTRACT

A nebulizer is connected to an inspiratory air line of a ventilator and forms an air path along a longitudinal axis through which air is delivered to a patient from a ventilator. A nebulizer nozzle is carried within the nebulizer housing and aligned with the longitudinal axis through which gas is mixed with medication and discharged into the air path. A suction line extends from the discharge end of the nebulizer nozzle through the outer surface of the nebulizer housing to a medication receiver that draws medication upward from a medication container contained within the medication receiver and mixes the medication with gas passing through the nebulizer nozzle and nebulizes the medication for discharge into the air path to a patient.

### FIELD OF THE INVENTION

The present invention relates to medical ventilators used to assist breathing in a patient, and more particularly, this invention relates to nebulizers used with mechanical and other medical ventilators.

### BACKGROUND OF THE INVENTION

Medical ventilators mechanically move breathable air into and out of a patient's lungs and provide the mechanism of breathing for the patient, who typically is physically unable to breathe or is breathing insufficiently. Positive pressure ventilators are common where air or another gas mixture is forced by pressure into the trachea using an endotracheal tube that rests in the trachea and usually held in position by an inflatable balloon positioned at the end of the endotracheal tube, which allows the endotracheal tube to sit securely in the airway. The positive pressure ventilator works by increasing the patient's airway pressure through the endotracheal tube or in some examples, through a tracheostomy tube that allows the positive pressure air to flow into the airway until the cyclical ventilator pressure imparting the breath is terminated. The elastic recoil of the chest wall and lungs force the breath out using as a passive exhalation. Other mechanical ventilators include transport ventilators, intensive care ventilators, neonatal ventilators and the ubiquitous positive airway pressure ventilator (PAPA) as a form of non-invasive ventilation used at home for treatment of sleep apnea or COPD (Chronic Obstructive Pulmonary Disease).

Ventilators are often used during surgery when a patient is under anesthesia, or suffering a debilitating disease or has some other chronic condition that impairs the patient's lung function such that mechanical ventilation is required to help the patient breathe. Such debilitating problems may result from pneumonia, COPD, upper spinal chord injuries, polio, Amyotrophic Lateral Sclerosis (ALS), brain injury, stroke, drug overdose, or other diseases that affect the nerves and muscles involved in breathing.

To aid in a patient's recovery or for administering medication to a patient who may not be able to ingest or swallow such drugs, aerosol drugs are commonly administered to the patients during mechanical ventilation. The ventilator circuit may incorporate jet nebulizers or similar nebulizer devices that nebulize medication for delivery to a patient through a heated/humidified ventilator circuit. These devices are often placed in the manifold position or other locations within the ventilator circuit to provide a patient with a nebulized medication in an efficient manner. Even slight improvements in nebulizer design would be beneficial to enhance the positive effects of the ventilation.

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

A nebulizer, in accordance with a non-limiting example, includes a tubular nebulizer housing having an outer surface and opposing ends defining an inlet and outlet configured to be connected in line with an inspiratory air line of a ventilator and forming an air path along a longitudinal axis through which air is delivered to a patient from a ventilator. A nebulizer nozzle is carried within the nebulizer housing and aligned with the longitudinal axis and has a gas intake end and a gas discharge end through which gas is mixed with medication and discharged into the air path. A medication receiver is carried on the outer surface of the nebulizer housing proximal to the nebulizer nozzle and formed to receive a medication container. A suction line extends from the discharge end of the nebulizer nozzle through the outer surface of the nebulizer housing into the medication receiver and draws medication upward from a medication container received within the medication receiver and mixes the medication with gas passing through the nebulizer nozzle and nebulizes the medication for discharge into the air path to a patient.

In one example, the nebulizer nozzle is formed as a venturi that is horizontally oriented when the nebulizer is connected into a ventilator inspiratory air line. A gas inlet line, in one example, is connected to the gas intake end of the nebulizer nozzle and extends through the outer surface of the nebulizer housing. In yet another example, the nebulizer housing includes first and second tubular housing members that are longitudinally aligned and connected together. One of the tubular housing members carries the nebulizer nozzle, medication receiver and suction line.

In yet another example, the medication receiver is formed as a cylindrical wall member carried on the outer surface of the tubular nebulizer housing. The suction line extends into the area defined within the cylindrical wall member. A medication container is received within the cylindrical wall member of the medication receiver and dimensioned to hold medication that is drawn upward through the suction line. A twist lock slot is formed in the cylindrical wall member and a lock protrusion is formed on the medication container that is received in the twist lock slot to lock the medication container within the medication receiver. A valve communicates with the gas intake end of the nebulizer nozzle and is configured to actuate a flow of gas through the nebulizer nozzle during each inspiratory phase of the respiratory cycle of the patient. A low pressure mixing chamber may be formed at the gas discharge end of the nebulizer nozzle. A continuous pressure is provided at the gas intake end. In an example, at standard temperature and pressure (STP), a differential pressure results in no medication being drawn upward through the suction line for nebulization until a predetermined negative inspiratory pressure is created such as occurs during the inspiratory phase of the patient's respiratory cycle.

In yet another example, a ventilator system is disclosed and includes a ventilator and an inspiratory air line connected to the ventilator through which pressurized air is provided to a patient from the ventilator. An expiratory air line is connected to the ventilator. A nebulizer as described above, for example, is connected within the inspiratory air line. A humidifier may be connected within the inspiratory air line and the nebulizer connected within the air line between the ventilator and the humidifier or connected within the expiratory air line between a patient and the humidifier.

A method aspect is also disclosed of delivering a nebulized medication to a ventilated patient by connecting the nebulizer as described above in-line with the inspiratory air line and connected to a ventilator through which pressurized air is provided to a patient. Gas is passed through the gas intake end of the nebulizer nozzle, and thus, medication is drawn upward from the medication container within the medication receiver and mixes the medication with the gas to nebulize the medication and discharge it into the air path to a patient.

### BRIEF DESCRIPTION OF THE DRAWINGS

Other objects, features and advantages of the present invention will become apparent from the detailed description of the invention which follows, when considered in light of the accompanying drawings in which:

FIG. 1 is a fragmentary, perspective view of a patient who requires assisted breathing and is on a ventilator, and showing the inspiratory and expiratory air lines connected to the ventilator, an endotracheal tube extending through the patient's mouth into their windpipe, and a nasogastric/orogastric (Ng/Og) tube passing through the patient's nose into the stomach in accordance with a non-limiting example.

FIG. 2 is an enlarged isometric view of the nebulizer and a coiled air line connected to the nebulizer in accordance with a non-limiting example.

FIG. 3 is a side elevation view of the nebulizer in accordance with a non-limiting example.

FIG. 4 is a front elevation view of the nebulizer taken in the direction of arrow 4 in FIG. 3 in accordance with a non-limiting example.

FIG. 5 is a sectional view of the nebulizer taken along line 5-5 of FIG. 4 in accordance with a non-limiting example.

FIG. 6 is an exploded, isometric view of the nebulizer and showing the medication container removed from the medication receiver in accordance with a non-limiting example.

FIG. 7 is a fragmentary sectional view of the nebulizer shown in FIG. 6 but showing the medication container inserted within the medication receiver.

FIG. 8 is a sectional view of another embodiment of the nebulizer showing a nebulizer nozzle that operates when a negative inspiratory pressure is created during the inspiratory phase of the patient's respiratory cycle.

### DETAILED DESCRIPTION OF THE PREFERRED EMBODIMENTS

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

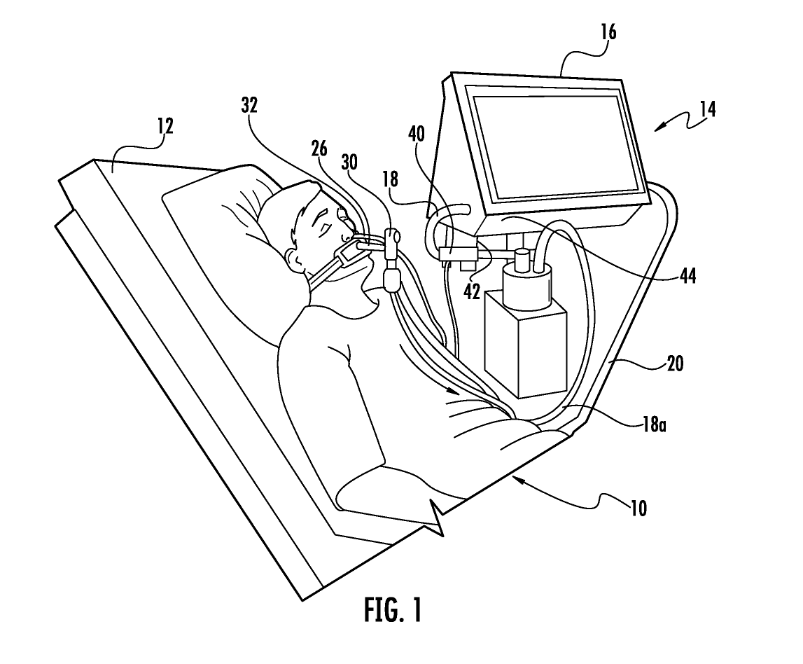

FIG. 1 is a fragmentary, perspective view showing a patient 10 in a hospital or other medical setting and lying inclined on a bed 12 and mechanically assisted in spontaneous breathing using a mechanical ventilation system indicated generally at 14 that includes a ventilator 16 and an inspiratory air line 18 connected to the ventilator through which pressurized air is provided to a patient from the ventilator. An expiratory air line 20 returns air and is connected back into the ventilator 16. As illustrated, the inspiratory air line is passed through a humidifier 22 that warms and moistens the air for delivery to the patient through an endotracheal tube 26 that extends through the patient's mouth into the windpipe. The endotracheal tube 26 connects to the ventilator 16 and its inspiratory line 18 via an endotracheal coupler 30 as illustrated. The expiratory air line 20 extends from the endotracheal coupler 30 back to the ventilator as part of the return air path.

In this example, a nasogastric/orogastric (Ng/Og) tube 32 extends through the patient's nose and into the stomach and provides different medical functions, including feeding the patient through the tube in one example and sucking stomach contents when necessary. An example of a Ng/Og tube 32 that may be used as illustrated includes those Ng/Og tubes disclosed in commonly assigned U.S. Pat. No. 8,597,184 and U.S. Patent Publication Nos. 2011/0046653 and 2011/0040211, the disclosures which are hereby incorporated by reference in their entirety. Those particular examples include a nasogastric/orogastric (Ng/Og) airway protection device that includes an inflatable esophageal cuff that protects the patient's airway from reflux or other stomach contents that may be regurgitated. That disclosed Ng/Og tube also provides the capability to induce an involuntary reflex cough test by imparting an irritant such as tartaric acid into the airway through a nebulizer lumen. The nebulizer 40 in accordance with a non-limiting example is shown connected into the inspiratory air line 18 of the ventilator 16 and horizontally configured. It is supported on a nebulizer support frame 42 that connects onto the ventilator housing 44.

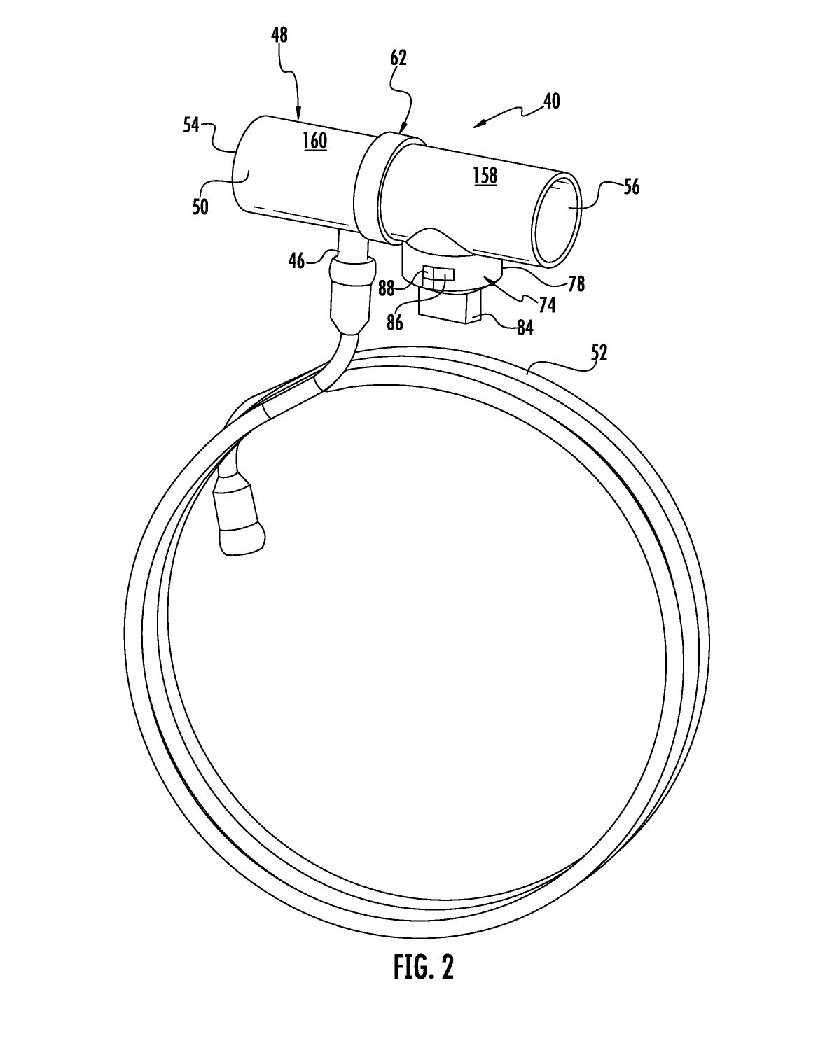

FIG. 2 is an enlarged fragmentary view of the nebulizer 40 showing a gas inlet line 46 that is connected to the tubular nebulizer housing 48 at its outer surface 50 and connects into an air source such as an air line 52 as illustrated in this example. The nebulizer 40 includes the tubular nebulizer housing 48 having its outer surface 50 and opposing ends defining an inlet 54 and outlet 56 that are both connected in-line to the inspiratory air line and form an air path 51 along its longitudinal axis through which air is delivered to a patient 10 from the ventilator 16. The nebulizer and internal components can be formed from various materials, including injection molded plastic. The nebulizer 40 is constructed to be easily replaced in-line to the inspiratory air line 18. This is advantageous because the nebulizer 40 often must be replaced and cleaned since albuterol and other medications can clog nebulizer components and any nozzle assemblies.

As illustrated, the nebulizer 40 is connected within the inspiratory air line 18 between the ventilator 16 and the humidifier 22 in the example shown in FIG. 1. In other embodiments, however, the nebulizer 40 could be connected within the inspiratory air line 18 between a patient 10 and the humidifier 22, for example, the location illustrated generally at lea corresponding to a midsection of the air line from the humidifier 22 to the endotracheal coupler 30 as shown in FIG. 1.

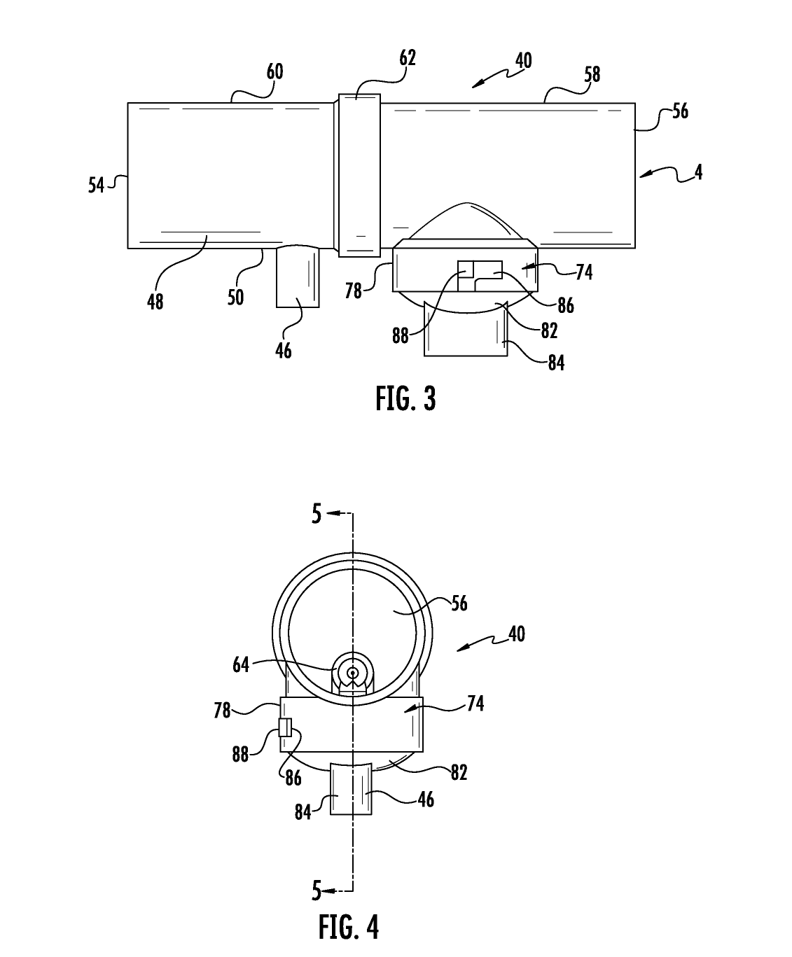

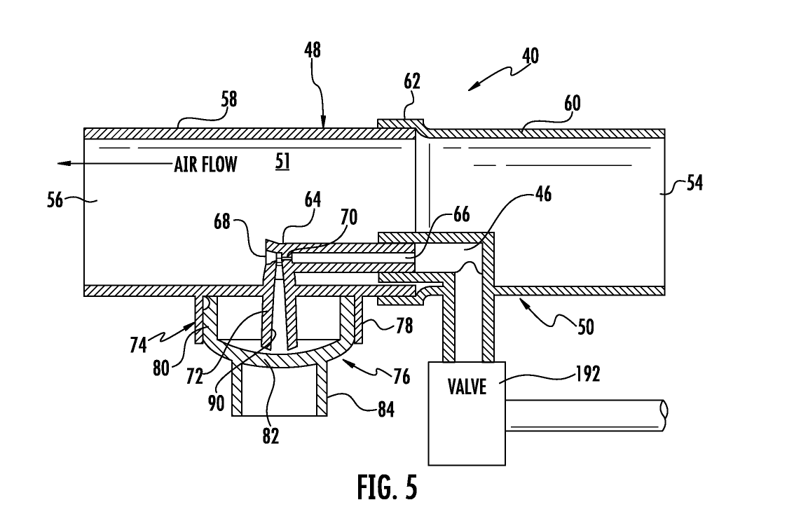

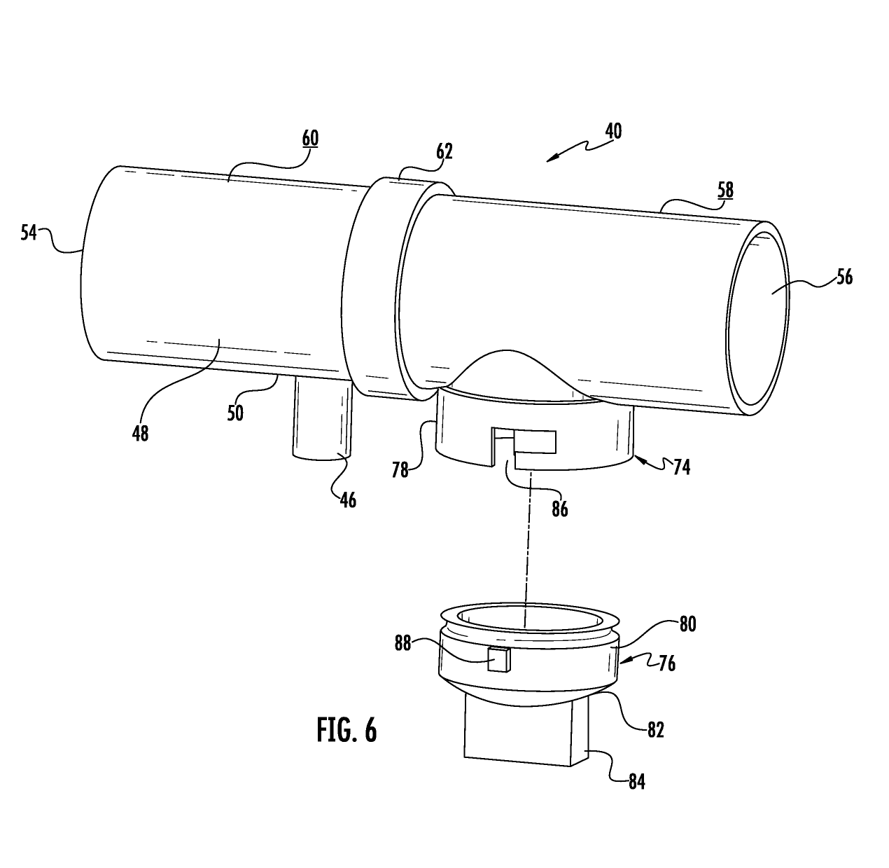

Referring now to FIGS. 3-5, there is shown greater details of the nebulizer 40. As illustrated, the nebulizer 40 is formed from first and second tubular housing members 58,60 as best shown in FIG. 6 that are longitudinally aligned and connected together. The first tubular housing member 58 is a straight tube at its end where it connects and the second tubular housing member 60 includes a flanged end 62 that receives the first tubular housing member 58 in a frictional fit and may be secured together at that point by an adhesive, threads or other means. This tubular nebulizer housing 48 formed from the first and second tubular housing members 58,60 has its outer surface 50 and opposing ends defining the inlet and outlet 54,56 of the nebulizer 40 that are connected in-line with the inspiratory air line 18 of the ventilator 16 and form an air path along its longitudinal axis through which air is delivered to a patient from the ventilator as shown in FIG. 1.

A nebulizer nozzle 64 is carried within the nebulizer housing 44, and in the example shown in FIG. 5, is aligned with the longitudinal axis defined by the nebulizer housing 44. The nebulizer nozzle 64 includes a gas intake end 66 and a gas discharge end 68 through which gas is mixed with the medication and discharged into the air path 51 defined by the internal cylindrical portion of the tubular nebulizer housing 48. The nebulizer nozzle 64 as illustrated is supported and aligned with the longitudinal axis formed by the tubular nebulizer housing 48 and includes a shorter venturi segment 70 at its gas discharge end 68 and connects to a suction line 72 that extends from the gas discharge end of the nebulizer nozzle 64 through the outer surface 50 of the nebulizer housing 48 such that the nebulizer nozzle and its gas intake end 66 is supported along the longitudinal axis within the tubular nebulizer housing.

A medication receiver 74 is formed on the outer surface 50 of the nebulizer housing 48 proximal to the nebulizer nozzle 64 and formed to receive a medication container 76 as illustrated, such that the suction line 72 extends from the gas discharge end 68 of the nebulizer nozzle 64 through the outer surface 50 of the nebulizer housing to the medication receiver 74 and draws medication upward from the medication container 76 inserted within the medication receiver and mixes the medication with gas passing through the nebulizer nozzle and nebulizes the medication for discharge into the air path 51 to a patient.

In this example, the medication receiver 74 is formed as a cylindrical wall member 78 positioned on the outer surface of the tubular nebulizer housing 48 proximal to the suction line 72 that extends through the outer surface 50 of the tubular nebulizer housing 48. The suction line 72 extends into the area (or volume) defined within the cylindrical wall member 78. The medication container 76 is received within the cylindrical wall member 78 of the medication receiver and is dimensioned to hold medication that is drawn upward through the suction line 72 that extends downward into that area or volume defined by the cylindrical wall of the medication receiver 74.

In this example, the medication container 76 is cup-shaped with cylindrical wall 80 and a curved or cup-shaped bottom 82 as best shown in FIG. 6, which holds the medication and includes a rectangular twist tab 84 at its bottom so that the tab may be grasped by a user, patient or doctor and insert the medication container 76 upward into the medication receiver 74 formed by the cylindrical wall member 78. In this example, a twist lock slot 86 is formed in the cylindrical wall member 7B as illustrated and a lock protrusion 88 is formed on the outer surface of the cylindrical wall 80 forming the medication container. The lock protrusion 88 is received in the twist lock slot 86 so that when the medication container is inserted upward into the medication receiver 74 and turned, the lock protrusion 88 extends into the horizontal portion of the twist lock slot 86 and locked into place. The medication container 76 may contain different drugs such as albuterol as an example or other drugs necessary for nebulization to the patient that is mechanically ventilated. The medication receiver and container can be designed similar to those disclosed in commonly assigned U.S. Pat. Nos. 7,712,466 and 7,726,306, the disclosures which are hereby incorporated by reference in their entirety.

As illustrated, the nebulizer nozzle 64 is aligned with the longitudinal axis and has a gas intake end 66 and a gas discharge end 68 through which the gas is mixed with the medication drawn upward through the suction line 72 and discharged into the air path as illustrated. The nebulizer nozzle 64 is formed as a venturi 70 in the example of FIG. 5 and it is horizontally oriented when the nebulizer is connected into the ventilator inspiratory air line 18 as shown in FIG. 1. Although a small venturi is disclosed, the venturi may be formed and angled from the gas discharge end 68 back to the gas intake end 66. In the example illustrated, the gas inlet line 46 is connected to the gas intake end 66 and to the nebulizer nozzle 64 and extends through the outer surface 50 of the nebulizer housing 48 as illustrated and carried by the second tubular housing member 60. When the first and second tubular housing members 58,60 are coupled together, the gas inlet line 46 connects into the gas intake end 66 of the nebulizer nozzle. In the example illustrated, the nebulizer nozzle 64 has an outer diameter that forms a frictional fit with the inner diameter of the gas inlet line 46 to form a press fit and aid in holding the two members together. The suction line 72 may also be formed with an internal venturi 90 as illustrated and is dimensioned to work in conjunction with the nebulizer nozzle venturi to nebulize a predetermined amount of medication and create a desired nebulization pattern.

In the example shown in FIG. 5, a gas valve 92 communicates with the gas intake end 66 of the nebulizer nozzle, and in this example, it is an external valve connected into the gas inlet line 46 and actuates a flow of gas through the nebulizer nozzle during each inspiratory phase of the respiratory cycle of a patient. In this example, as the ventilator 16 operates to provide positive air flow pressure on an intermittent basis, each time the ventilator pressurizes the inspiratory air line 18 to mechanically ventilate the patient, the gas valve 92 actuates the air flow through the nebulizer nozzle 64 and provides nebulization of the medication contained within the medication container 76 such that it is drawn into the patient during the inspiratory phase of the respiratory cycle of the patient. In this example, the nebulizer 40 is positioned in the inspiratory air line 18 between the humidifier 22 connected within the inspiratory air line and the ventilator 16. This has been found to be an advantageous location, but the nebulizer may also be connected within the inspiratory air line between a patient and the humidifier.

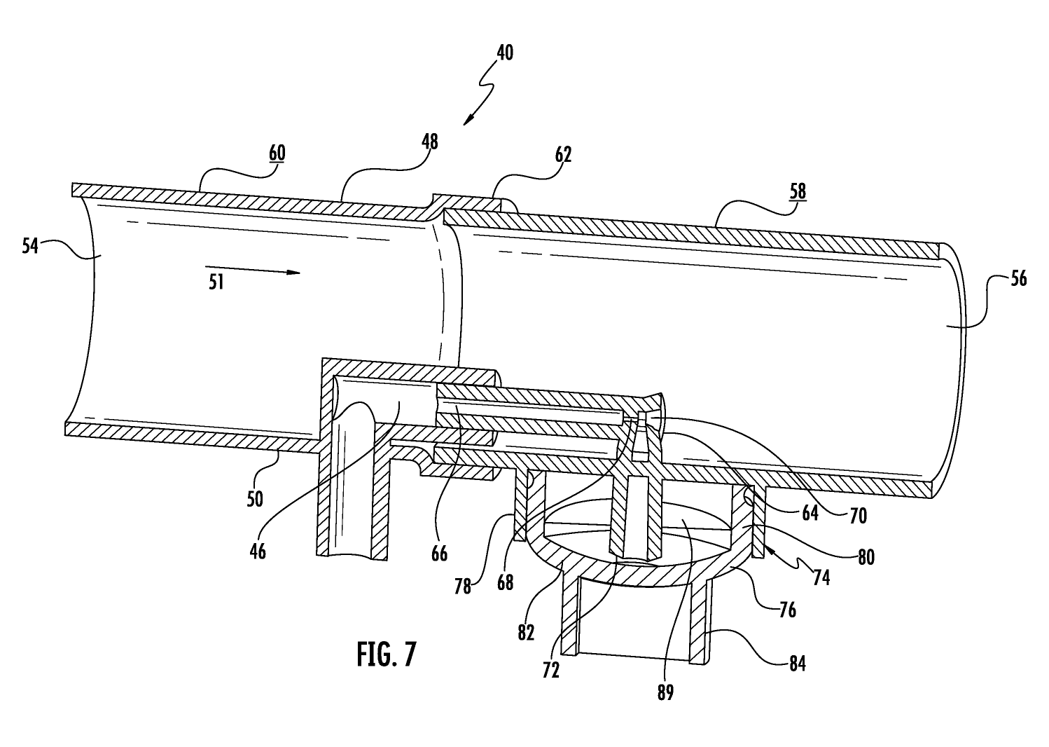

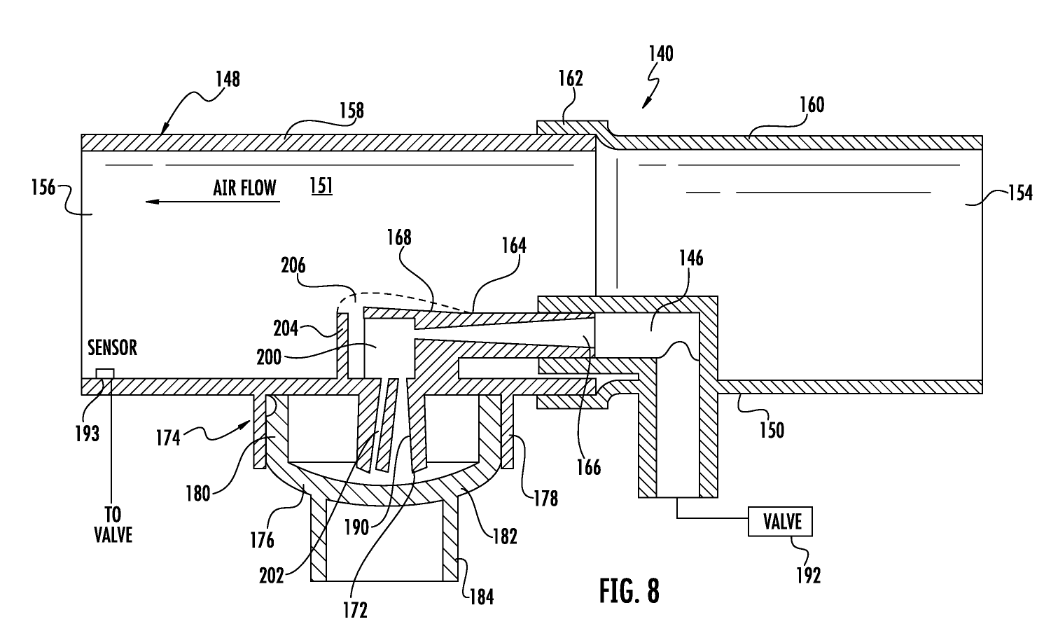

FIG. 6 is an enlarged perspective isometric view of the nebulizer 40 showing the medication container 76 removed from the medication receiver 74 and showing its cylindrical wall 80 and cup-shaped bottom 82 that holds the medication. The medication may be stored within the cup-shaped bottom 82 and covered with a seal or other plastic covering to protect the medication until it is ready to be used and nebulized. When the nebulizer is prepared for use, the medication container 76 is moved upward and the lock protrusion 88 formed on the cylindrical wall 80 of the medication container is received within the twist lock slot 86, the suction line 72 will break the seal protecting the medication. The suction line 72 then extends into the cup-shaped bottom 82 of the medication container as shown in FIG. 5 and be received within the medication that is stored within the medication container. FIG. 7 shows an example level of medication in the cup-shaped bottom 82 and a portion of the seal 89 that had been broken by the suction line 72.

FIG. B is another embodiment of the nebulizer showing a different nebulizer nozzle, but still configured as a venturi, and also having a low pressure mixing chamber formed at the gas discharge end of the nebulizer nozzle. For purposes of description, similar elements in this example nebulizer of FIG. 8 are given the same reference numerals as before, except given in the 100 series.

Continuous pressure is provided at the gas intake end 166 wherein at standard temperature and pressure (STP), a differential pressure results in no medication being drawn upward through the suction line 172 for nebulization until a predetermined negative inspiratory pressure is created during the inspiratory phase of the patient's respiratory cycle. In this example, the nebulization is drawn upward through the suction line 172 into the mixing chamber 200 during that time when the ventilator is in a positive pressure cycle that is equivalent to the inspiratory phase of the patient's respiratory cycle such as when a negative inspiratory pressure is created when a patient is breathing in.

An example of a similar nebulizer structure is disclosed in commonly assigned U.S. patent application Ser. No. 13/353,611 and Ser. No. 13/799,196, the disclosures which are hereby incorporated by reference in their entirety. This embodiment includes a much longer and gradual taper to the venturi 170 as illustrated and a secondary suction line 202 that interoperates with the mixing chamber. A baffle or diffuser 204 is located proximal to the mixing chamber.

The suction line 170 acts as a primary suction line and the secondary suction line 202 aids in drawing nebulized medication that drops down before discharge through the nebulizer outlet due to the further break-up from the diffuser or baffle 204. Further details of the operation of such a nebulizer is explained in the incorporated by reference '611 and '196 applications. Although not illustrated in detail, the structure in FIG. 8 together with the baffle or diffuser 204 forms a rainfall chamber 206 in which the nebulizer nozzle 164 and low pressure mixing chamber 200 are contained. The nebulizer 140 can be activated at the inspiratory pressure referring to the pressure generated by the ventilator and from about −3 cmH₂O to about −52 cmH₂O.

It is also possible to use an air flow sensor 193 to measure air flow and send a signal back to the gas valve 192 and either actuate or modify air flow through the nebulizer nozzle 164. The air flow sensor 193 could be positioned at the nebulizer outlet and sense inspiratory pressure. The droplet sizes of the nebulized medication can vary and can be small or less than 0.1 microns. The air flow can vary from 2 liters up to 15 liters air flow in one non-limiting example.

It is also possible to use the nebulizer in both a pressure limited ventilation or the volume cycled ventilation. During a pressure limited ventilation, the nebulizer is driven continuously. The nebulizer can also be operated under pulsed operation such as disclosed in U.S. patent application Ser. No. 14/166,882 filed Jan. 29, 2014, the disclosure which is hereby incorporated by reference in its entirety. This nebulizer used with a ventilator provides a horizontal venturi nozzle (HVN) and directly nebulizes in the inhalation circuit of the ventilator. There is less distance to the deposition area and little condensation with low residual volume. Small volume but high concentrated medications can be applied.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A nebulizer, comprising:
a tubular nebulizer housing having an outer surface and opposing ends defining an inlet and outlet configured to be connected to an inspiratory air line of a ventilator and forming an air path along a longitudinal axis through which air is delivered to a patient from a ventilator;
a nebulizer nozzle carried within the nebulizer housing and aligned with the longitudinal axis and having a gas intake end and a gas discharge end through which gas is mixed with medication and discharged into the air path;
a medication receiver on the outer surface of the nebulizer housing proximal to the nebulizer nozzle and formed to receive a medication container; and
a suction line extending from the discharge end of the nebulizer nozzle through the outer surface of the nebulizer housing to the medication receiver that draws medication upward from a medication container contained within the medication receiver and mixes the medication with gas passing through the nebulizer nozzle and nebulizes the medication for discharge into the air path to a patient.

2. The nebulizer according to claim 1 wherein the nebulizer nozzle is formed as a venturi that is horizontally oriented when the nebulizer is connected into a ventilator inspiratory air line.

3. The nebulizer according to claim 1 and further comprising a gas inlet line connected to the gas intake end of the nebulizer nozzle and extending through the outer surface of the nebulizer housing.

4. The nebulizer according to claim 1 wherein said nebulizer housing comprises first and second tubular housing members that are longitudinally aligned and connected together, wherein one of the tubular housing members carries the nebulizer nozzle, medication receiver and suction line.

5. The nebulizer according to claim 1 wherein said medication receiver comprises a cylindrical wall member formed on the outer surface of the tubular nebulizer housing, and said suction line extends into the area defined within the cylindrical wall member.

6. The nebulizer according to claim 5 further comprising a medication container received within the cylindrical wall member of the medication receiver and dimensioned to hold medication that is drawn upward through the suction line.

7. The nebulizer according to claim 6 and further comprising a twist lock slot formed in the cylindrical wall member and a lock protrusion formed on the medication container that is received in twist lock slot to lock the medication container within the medication receiver.

8. The nebulizer according to claim 1 further comprising a valve communicating with the gas intake end of the nebulizer nozzle and configured to actuate a flow of gas through the nebulizer nozzle during the inspiratory phase of the respiratory cycle of the patient.

9. The nebulizer according to claim 1 comprising a low pressure mixing chamber formed at the gas discharge end of the nebulizer nozzle and wherein continuous pressure is provided at the gas intake end, wherein at standard temperature and pressure (STP), a differential pressure results in no medication being drawn upward through the suction line for nebulization until a predetermined negative inspiratory pressure is created during the inspiratory phase of the patient's respiratory cycle.

10. A ventilation system comprising:
a ventilator and an inspiratory air line connected to the ventilator through which pressurized air is provided to a patient from the ventilator and an expiratory air line connected to the ventilator;
a nebulizer connected within the inspiratory air line, comprising:
a tubular nebulizer housing having an outer surface and opposing ends defining an inlet connected to the inspiratory air line and an outlet connected to the inspiratory air line, and forming an air path along a longitudinal axis through which air is delivered to a patient from the ventilator;
a nebulizer nozzle carried within the nebulizer housing and aligned with the longitudinal axis and having a gas intake end and a gas discharge end through which gas is mixed with medication and discharged into the air path;
a medication receiver on the outer surface of the nebulizer housing proximal to the nebulizer nozzle and formed to receive a medication container; and
a suction line extending from the discharge end of the nebulizer nozzle through the outer surface of the nebulizer housing to the medication receiver that draws medication upward from a medication container contained within the medication receiver and mixes the medication with gas passing through the nebulizer nozzle and nebulizes the medication for discharge into the air path to a patient via the inspiratory air line of the ventilator.

11. The ventilation system according to claim 10 and further comprising a humidifier connected within the inspiratory air line.

12. The ventilation system according to claim 11 wherein said nebulizer is connected within the inspiratory air line between the ventilator and the humidifier.

13. The ventilation system according to claim 11 wherein said nebulizer is connected within the inspiratory air line between a patient and the humidifier.

14. The ventilation system according to claim 10 wherein the nebulizer nozzle is formed as a venturi that is horizontally oriented when the nebulizer is connected within the inspiratory air line.

15. The ventilation system according to claim 10 and further comprising a gas inlet line connected to the gas intake end of the nebulizer nozzle and extending through the outer surface of the nebulizer housing.

16. The ventilation system according to claim 10 wherein said nebulizer housing comprises first and second tubular housing members that are longitudinally aligned and connected together, wherein one of the tubular housing members carries the nebulizer nozzle, medication receiver and suction line.

17. The ventilation system according to claim 10 wherein said medication receiver comprises a cylindrical wall member formed on the outer surface of the tubular nebulizer housing, and said suction line extends into the area defined within the cylindrical wall member.

18. The ventilation system according to claim 17 further comprising a medication container received within the cylindrical wall member of the medication receiver and dimensioned to hold medication that is drawn upward through the suction line area.

19. The ventilation system according to claim 18 and further comprising a twist lock slot formed in the cylindrical wall member and a lock protrusion formed on the medication container that is received in twist lock slot to lock the medication container within the medication receiver.

20. The ventilation system according to claim 10 further comprising a valve communicating with the gas intake end of the nebulizer nozzle and configured to actuate a flow of gas through the nebulizer nozzle during the inspiratory phase of the respiratory cycle of the patient.

21. The ventilation system according to claim 10 comprising a low pressure mixing chamber at the gas discharge end of the nebulizer nozzle and wherein continuous pressure is provided at the gas intake end, wherein at standard temperature and pressure (STP), a differential pressure results in no medication being drawn upward through the suction line for nebulization until a predetermined negative inspiratory pressure is created during the inspiratory phase of the patient's respiratory cycle.

22. A method of delivering a nebulized medication to a ventilated patient, comprising:
connecting a nebulizer in-line with an inspiratory air line connected to a ventilator through which pressurized air is provided to a patient, the nebulizer comprising,
a tubular nebulizer housing having an outer surface and opposing ends defining an inlet and outlet connected with the inspiratory air line of the ventilator and forming an air path along a longitudinal axis;
a nebulizer nozzle carried within the nebulizer housing and aligned with the longitudinal axis and having a gas intake end and a gas discharge end;
a medication receiver on the outer surface of the nebulizer housing proximal to the nebulizer nozzle and formed to receive a medication container;
a suction line extending from the discharge end of the nebulizer nozzle through the outer surface of the nebulizer housing to the medication receiver; and
passing a gas through the gas intake end of the nebulizer nozzle and drawing medication upward from a medication container contained within the medication receiver and mixing the medication with the gas passing through the nebulizer nozzle to nebulize the medication and discharge it into the air path to a patient.

23. The method according to claim 22 comprising actuating a flow of gas through the nebulizer nozzle during the inspiratory phase of the respiratory cycle of the patient.

24. The method according to claim 22 comprising connecting a humidifier within the inspiratory air line.

25. The method according to claim 24 comprising connecting the nebulizer within the inspiratory air line between the ventilator and the humidifier.

26. The method according to claim 24 comprising connecting the nebulizer within the inspiratory air line between a patient and the humidifier.

27. The method according to claim 22 comprising the forming the nebulizer nozzle as a venturi that is horizontally oriented when the nebulizer is connected within the inspiratory air line.
